(19) United States
(12) Patent Application Publication
(10) Pub. No.: US 2014/0000630 A1
(43) Pub. Date: Jan. 2, 2014

(54) Magnetic Imaging Device To Inventory Human Brain Cortical Function

(71) Applicant: John P. Ford, Unadilla, NY (US)

(72) Inventor: John P. Ford, Unadilla, NY (US)

(21) Appl. No.: 13/804,070

(22) Filed: Mar. 14, 2013

Related U.S. Application Data
(60) Provisional application No. 61/666,171, filed on Jun. 29, 2012.

(51) Int. Cl. A61B 5/04; A61G 13/12

(52) U.S. Cl. CPC A61B 5/04008; A61G 13/12
USPC 128/845; 600/409

(57) ABSTRACT

Systems and methods detect electrical activity in the human brain in the form of the generated magnetic fields and map the magnetic field strength to the surface of the cerebral cortex. By mapping the measured magnetic fields to the sulcal-defined gyrus of the cerebral cortex rather than to its three-dimensional volume, the data complexity and the resulting image are dramatically reduced. The device allows an inventory of brain function, including, but not limited to, vision, sound, sensory, and cognition, with the sensors placed over the corresponding region or regions of interest of the brain.

### REFERENCE TO RELATED APPLICATIONS

This application claims one or more inventions which were disclosed in Provisional Application Number 61/666,171, filed Jun. 29, 2012, entitled “MAGNETIC IMAGING DEVICE TO INVENTORY HUMAN BRAIN CORTICAL FUNCTION”. The benefit under 35 USC §119(e) of the United States provisional application is hereby claimed, and the aforementioned application is hereby incorporated herein by reference.

### BACKGROUND OF THE INVENTION

1. Field of the Invention

The invention pertains to the field of medical imaging. More particularly, the invention pertains to systems and methods of detecting electrical activity in the brain.

2. Description of Related Art

The adult human cerebral cortex has an average volume in the range of about 870 to 970 cm³ and includes about 10¹⁰ neurons forming about 10¹⁴ neural connections. Even though neurons constitute only about 10% of the brain cell population, this complexity presents a huge imaging problem. A functional three-dimensional image of a volume with this complexity would be almost impossible both to display and to understand. Current cerebral cortex imaging approaches do not image neural function directly. Functional magnetic resonance imaging (fMRI) images blood flow, and positron emission tomography (PET) images metabolism. fMRI is sensitive on a seconds time frame, but normal events in the brain occur in a millisecond time frame. For example, imaging neural transmission in real time allows for assessment of the efficacy of drug interventions for medical conditions affecting brain function.

A limitation for an electroencephalogram (EEG), which is sensitive to a millisecond time frame, is the problem of unpredictable signal attenuation by the tissues that surround the brain, a problem which does not exist for the magnetic field generated by the electric impulses in the brain. EEG signals do not attenuate predictably, however, such that both near and far signals are comingled (see, for example, Gallen et al., “Magnetoencephalography and Magnetic Source Imaging: Capabilities and Limitations”, Functional Neuroimaging, Vol. 5, pp. 227-249, 1995 and Hari, “The Neuromagnetic Method in the Study of the Human Auditory Cortex”, pp. 222-282 in Auditory Evoked Magnetic Fields and Electric Potentials, by Grandori et al. ed., Karger: Basel, Switzerland, 1990). This problem is particularly true of multiple current sources (e.g., both primary and secondary cortical sources).

A superconducting quantum interference device (SQUID) sensor includes a ring of one or more superconducting materials containing one or more Josephson junctions, which operate based on the Josephson effect. A superconductor is any material having no electrical resistance, but this property only occurs with certain materials below a critical temperature. A superconductor is also able to exclude a magnetic field, an attribute termed the Meissner effect. At a Josephson junction, a pair of superconductors is coupled by a weak link, which produces an indefinitely-long current without any applied voltage as a result of quantum tunneling of electron pairs termed Cooper pairs. The weak link may be a thin insulating barrier, a short section of non-superconducting metal, or a constriction that weakens the superconductivity at a point of contact. An external magnetic field changes the superconducting wave function of the SQUID sensor, which can be detected and quantified such that magnetic field strengths as weak as 10⁻¹⁵ Teslas (T) may be measured. The extreme sensitivity of SQUIDs makes them ideal for studies in biology, where biological processes produce magnetic fields in the range of 10⁻⁶ to 10⁻⁹ T.

Magnetoencephalography (MEG) is a non-invasive technique used to map brain activity based on detection by SQUID of magnetic fields produced by electrical activity in the brain and has been used to make inferences about neural activity inside brains (see, for example, Georgopoulos et al., “Synchronous Neural Interactions Assessed by Magnetoencephalography: A Functional Biomarker for Brain Disorders”, Journal of Neural Engineering, Vol. 4, pp. 349-355, 2007). Since SQUIDs have acquisition rates much higher than the highest temporal frequency of interest in the signals emitted by the brain (kHz), MEG gives good temporal resolution. A significant challenge in MEG, however, is the filtering out of environmental magnetic noise, which may be considerably higher (six or greater orders of magnitude) than the brain-generated magnetic fields of interest. Gallen et al. discuss the features and limitations of MEG in greater detail. In some cases, magnetic shielding is used to reduce the level of environmental magnetic noise reaching the SQUID sensors. In order for the entire brain to be evaluated, the subject wears a helmet containing about 300 SQUID sensors, which are configured to measure either an axial gradient or an off-diagonal gradient. Room shielding and the use of gradiometers eliminate the environmental noise magnetic signal, which is many orders of magnitude greater than the magnetic signal generated by the brain.

The reason that SQUID/MEG is virtually not used clinically today, despite a technology that is several decades old, derives from several problems:

First, magnetically-shielded rooms are very expensive.

Second, the cost of a 300-sensor SQUID is exorbitant, at a cost of about $10,000 per SQUID sensor.

Third, solving the virtually infinitely complex inverse problem (i.e., identifying the location of the relevant current sources) has not been accomplished and may not be possible.

There are at least three additional constraints to the use of MEG signals to image brain electrical activity:

1) A superconducting quantum interference device (SQUID) is a very sensitive magnetometer that is able to detect even a very weak magnetic field generated perpendicular to a current source but not magnetic fields generated by a radial current or dipole.

2) The strength of the generated magnetic field decays as the inverse square of the distance from a source of magnetism having “length”, called a dipole source, and as the inverse cube from a point source of magnetism.

3) The detection of the source of a magnetic field must be directed either to optimize the sensitivity or the localization of the magnetic source.

The detected SQUID signal represents a small subset of total current sources. The SQUID does not detect magnetic flux from radial current sources originating on the surface of the cortex. The SQUID also does not detect magnetic flux from oppositely-directed, and thus magnetically neutralized, current sources. Thus, a SQUID array optimized to localize superficial magnetic sources detects the neural currents tangential to the head surface.

The human brain has a corrugated surface with an average surface area of the cerebral cortex of about 2,400 cm² for adults. The invaginations in the brain cerebral cortex surface are termed sulci, and the smooth surfaces of the cerebral cortex between the sulci are termed gyri. The generated magnetic fields reflect the flux from the unique currents largely originating from cells lining the wall of the sulcal invaginations. Such a SQUID detects electrical signals from only a small subset (˜10⁻⁻³ or less) of the total neuron population. Because of the robust number of connections (10⁴ per neuron), the assumption is that any significant structural abnormality within the brain substance has a functional reflection in a map of topographical sulcal delimited gyral electrical activity. Evidence for this exists. Although the hippocampus is vital for memory creation, connections to the cortex are necessary for stable memory function (see, for example, Sweatt, “Creating Stable Memories”, Science, Vol. 331, pp. 869-870, 2011).

SQUID has already been used to detect evoked neural activity in the human cortex. An array of detectors with fixed locations on the human skull generates an image of the sensory-evoked neural activity of the brain. For example, stimulation of the finger creates a signal over the contralateral side of the brain in the region of the major sulcus overlying-region responsible for controlling the finger in the “homunculus”. Thus, the image sought is on a very small part of the brain. Alternatively, stimulation of the visual cortex (visual-evoked magnetic field) and auditory cortex (auditory-evoked magnetic field) have been mapped to the relevant cortical regions of the brain.

### SUMMARY OF THE INVENTION

The device generates a functional display of brain activity initially in a region of the cortex stimulated by an evoked potential. For example, a defined sound is produced and the response of the auditory cortex is detected and displayed such that the magnetic field generated at the acoustic gyrus is displayed. The pattern of intensity of response and timing of response is depicted in a single display. Variance from normal signal intensity and timing is evident. The output is displayed as a time lapse image of magnetic field intensity with no attempt to solve the “inverse problem”.

A similar inventory of brain function can be done for visual function, sensory motor function, and each cognitive function. In each case, the sensor array is situated over the brain region or regions relevant to the brain function being inventoried. The output of such an inventory, although the “inverse problem” is not solved, is highly useful. The magnitude and character of the output deviation from normal define the disease state and may be followed over time with therapeutic intervention.

The cost of the device is estimated to be about 10% the cost of alternative current instruments but with vastly superior information display characteristics.

### BRIEF DESCRIPTION OF THE DRAWINGS

FIG. 1 shows schematically a test subject in a movable patient support device in an embodiment of the present invention.

FIG. 2 shows schematically a top view of a sensor head with an array of SQUID sensors with the five surrounding sensors focused to an area about two to four centimeters below the central sensor in an embodiment of the present invention.

FIG. 3 shows a cross section of the SQUID sensor head of FIG. 2 along line 3-3 with the sensor head oriented to detect a magnetic field generated by electrical signals near a sulcus of a brain in an embodiment of the present invention.

FIG. 4a shows the formatting grid of relative field strength and latency of a single magnetic field compared to a result for the normal population in an embodiment of the present invention.

FIG. 4b shows a sample display of test results related to the sample test results of FIG. 4d on a formatting grid as in FIG. 4a.

FIG. 4c shows a sample display of test results of certain frequencies for the sample test results of FIG. 4d on a formatting grid as in FIG. 4a.

FIG. 4d shows a sample test result at a single time point from the SQUID system in an embodiment of the present invention.

### DETAILED DESCRIPTION OF THE INVENTION

To achieve adequate data homogenization in order to render the content of the collected data understandable without degrading it, in the current apparatus and methods the data collection is limited to neural transmission originating in the most superficial neurons lining the sulci of the relevant gyms of the human cortex. Also, the output is presented as a contour map and no attempt is made to determine the underlying dipole or current structure.

Systems and methods include a number of major simplifications over prior art systems and methods. First, the system preferably uses a single wire Faraday cage. The Faraday cage is a wire enclosure formed by a mesh of conducting material and blocks external static and non-static electric fields by canceling out their effects on the interior of the cage. The Faraday cage surrounds the human subject and SQUID equipment.

Second, far fewer SQUID detectors are required, which reduces the equipment cost. In some embodiments, about seven detectors are used. Third, a particular region of interest of the brain is identified as the subject of study (e.g., the superior temporal gyrus). Fourth, by having a movable patient support device and a fixed array of sensors, the region of interest can be precisely defined. The sensitivity of the SQUID device with a patient support device that is movable either manually or by a software program to optimize image quality can be equivalent to a static array of infinite sensor density. The support device is non-magnetic and non-paramagnetic (ideally completely of plastic components) to prevent any interference with the SQUID device. A fixed sensor apparatus is much cheaper, as elimination of the mitigation cost of the spill hazard of liquid helium in a movable Dewar is substantial. Conventional MEG systems cost $2 million or more, whereas the systems described herein may provide significantly better images of the cortical region of interest at one-tenth the equipment cost.

Systems and methods detect electrical activity in the human brain in the form of the magnetic fields generated by the electrical activity and map the magnetic field strength to the surface of the cerebral cortex. The sensor array is located over the cortical region controlling the function to be inventoried. For auditory evoked potential, the detector is placed over the superior temporal gyms to record initial response to a repeated sound stimulus. The patient support device is moved to refine the topological image quality. The contour maps of magnetic field intensity are collected over 500 msec after a defined stimulus (e.g., pitch, intensity, duration, and repetition). The data of magnetic field intensity and latency (see, for example, Knuutila et al., “Large-area low-noise seven-channel DC SQUID magnetometer for brain research”, Rev. Sci. Instrum., Vol. 58, pp. 2145-2156, 1987) of the test subject in response to a defined stimulus, once acquired, is compared to that of the idealized contour map of a “normal” population. By adjusting the machine settings, the display is transformed to show the latency of appearance of all the magnetic fields and the strength as a function of frequency of all the magnetic fields detected. The results are compared to the same magnetic fields in the normal population.

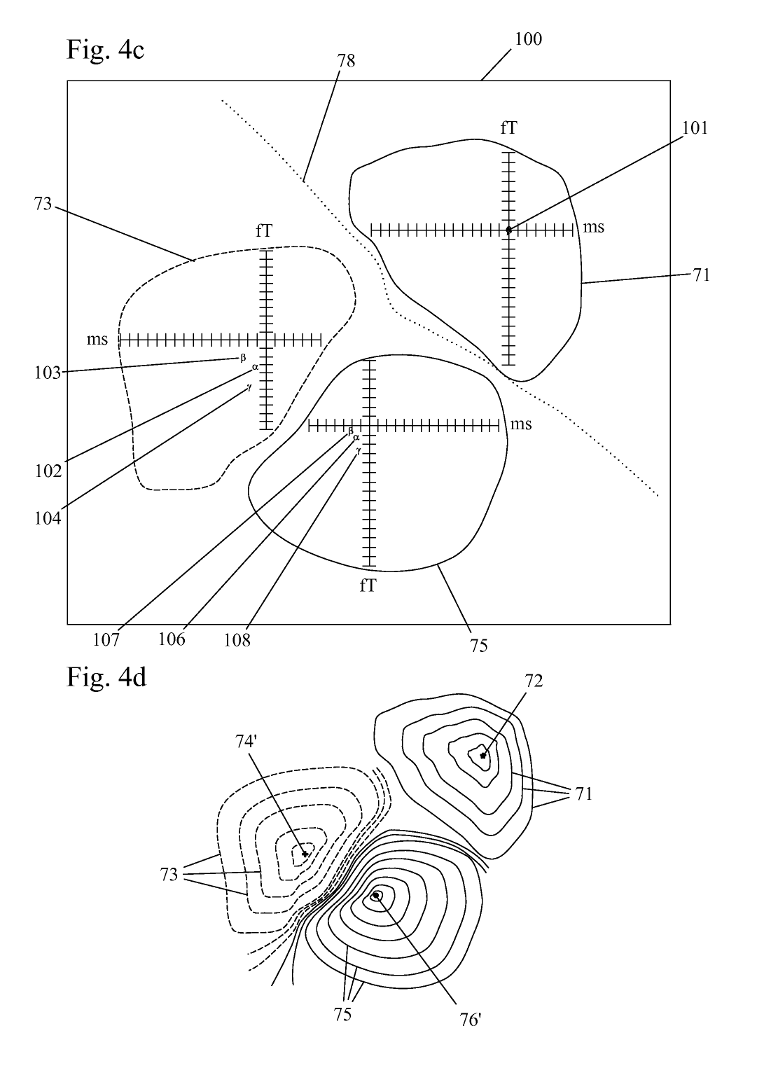

The strength maximum of each magnetic field relative to normal is displayed on the y-axis and the latency of appearance of the maximum of each magnetic field is displayed on the x-axis. The respective graphs are located within the limits of the respective contour map of the primary data image. If the strength and latency of the magnetic field match those of the “normal” population, a “point” is located at the intersection of the x- and y-axes (i.e., the origin). Changes in latency are noted by the “point” being located to the right of the origin if the latency is decreased compared to normal and to the left of the origin if the latency is increased. The displacement distance to the left or right of the “point” is proportional to the time difference from normal in msec (see FIG. 4a through FIG. 4c).

The field strength of each magnetic field is also represented within the respective field contour limits generated by the primary data. Stronger than normal magnetic fields displace the “point” up and weaker magnetic fields displace the “point” down. The displacement distance of the point “up” or “down” is a function of the difference in field strength from “normal”, measured in femtotesla (fT, see FIG. 4a through FIG. 4c).

After that topology display is complete and after 50 to 100 repetitions of the test, the image displayed for each of the approximately 10 ms snapshots of the 500 ms window displays either the raw data with signal power latency and frequency displayed by the point (raw data mode) or else the change of the point location reflecting the change from one or more prior tests or “normal” results (comparator mode). In the comparator mode, if the results are normal or if there is no change from the prior test, the point is located at the origin. Any change in the point location, reflecting a changed or abnormal result, is therefore visually obvious by not being located at the origin and may then be compared in a three-way test of the subject's prior test and a “normal” result to determine if the change moved the point closer to the origin (“normal” result) or farther away, i.e., whether the condition is worsening or improving.

Zamrini et al. (“Magnetoencephalography as a Putative Biomarker for Alzheimer's Disease”, International Journal of Alzheimer's Disease, Vol. 2011, Article ID 280289, 2011) identifies that one barrier to the use of MEG in the diagnosis and monitoring of the progression of Alzheimer's disease is the lack of software to process raw MEG data and provide a meaningful display of subject data. Such software may allow for the identification of patients with an intermediate level of cognitive function (“minimal cognitive impairment” or MCI) between that of normal individuals and that of patients with Alzheimer's disease.

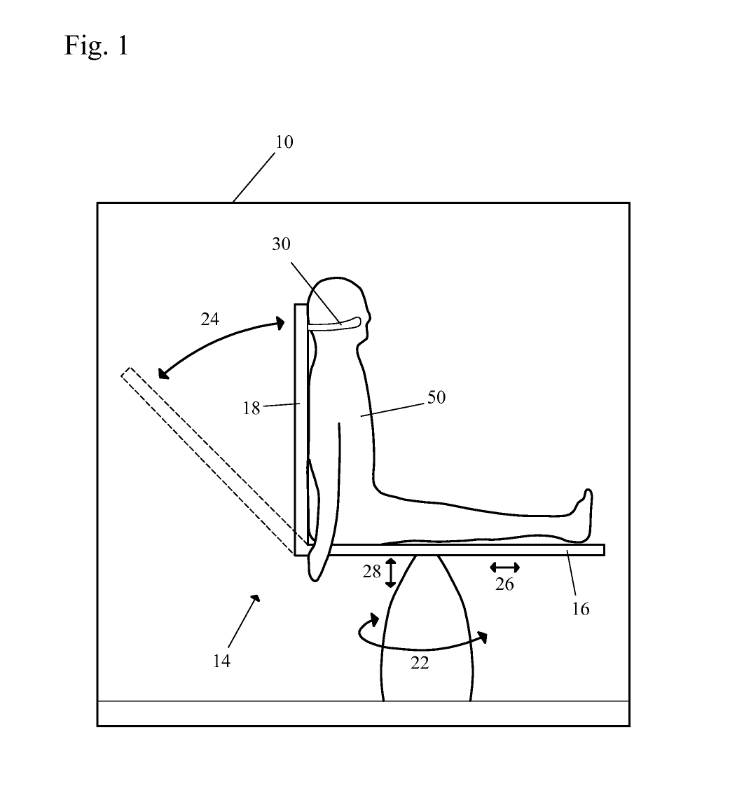

For patients with Alzheimer's disease, the 500 ms window after a test stimulus is known to show significant differences throughout this time interval between normal individuals and test patients with Alzheimer's disease (see, for example, FIG. 1 of Pekkonen et al., “Impaired preconscious auditory processing and cognitive functions in Alzheimer's disease”, Clinical Neurophysiology, Vol. 110, pp. 1942-1947, 1999).

The point may also reflect the signal for the entire frequency range (0-100 Hz) or for a narrower window or multiple windows. By convention, the component frequency bands of brain electrical waves are designated delta (δ0.5 to 4 Hz), theta (θ, 4 to 8 Hz), alpha (α, 8 to 13 Hz), beta (β, 13 to 30 Hz), gamma (γ, 30 to 48 Hz), and high gamma (Hγ, 49 to 100 Hz). The result with the device and software allows the band of interest to be displayed and represented by the corresponding Greek letter on the contour map (see FIG. 4c). The frequency band has also been shown to be important in distinguishing the MEG signals of normal individuals from those of Alzheimer's patients (see, for example, Berendse et al., “Magnetoencephalographic analysis of cortical activity in Alzheimer's disease: a pilot study”, Clinical Neurophysiology, Vol. 111, pp. 604-612, 2000). The location of the point reflecting a frequency band preferably has the same meaning in the contour map relative to prior studies as the point does reflecting the entire frequency range.

The sensor array may be placed over the inferior frontal gyrus to detect the “top down” response from the cortical executive region. The latter part of the 500 ms signal over the auditory cortex (see above) may likely also capture some of this information. Similar experiments have been done with EEG, but the limitations of EEG (see above) do not allow the distinction between the normal and the minimally conscious state (see Boly et al., “Preserved Feedforward But Impaired Top-Down Processes in the Vegetative State”, Science, Vol. 332, pp. 858-862, 2011). The same strategy is used for visual, sensory, motor, and cognitive inventory.

Of particular importance, the device generates a dynamic functional map as contrasted with current static maps. The images produced from evoked potentials, such as sensory stimulation of a body part, auditory evoked potentials, visual evoked potentials, and others, provide information that is not available by any other means, including, computed tomography (CT), MRI, or EEG.

The evoked response, which can take up to 500 ms, can have a complex structure of changes in the magnetic field of interest. In order to display these changes on a single display, the software of the present device displays the 0.5-s event over a variable but much longer time period preferably defined by the operator.

The detector array is preferably fixed at a predetermined angle with respect to vertical. The predetermined angle is preferably about 50° or less. In a preferred embodiment, the detector array is fixed at an angle of about 45° from vertical with five sensors at the points of a pentagon, each preferably about 2 cm from a central sixth sensor. Each sensor is preferably about 1.5 cm in diameter. The peripheral sensors are preferably aimed at a point about 2 cm below the central sensor. The device preferably includes a Dewar flask with a small liquid helium reservoir. The test subject preferably sits in a patient support device that is tiltable up to about 45° or 50° from vertical and rotatable at least 360°, similar to a dentist chair, but with precise control of the orientation and tilt of the patient support device. As mentioned previously, by having the patient support device move and having the sensor array fixed, the device cost is dramatically reduced. The precise location of the patient support device is communicated to the software developing the topological map. The patient support device preferably stabilizes the test subject's head by a cushioned support on each maxilla. The entire device is preferably housed in a Faraday cage to shield environmental magnetic flux. Such a device may be used anywhere and is expected to cost only about $200,000.

FIG. 1 shows a test subject seated in a patient support device 14. A Faraday cage 10 surrounds the test subject 50 and the patient support device 14 to block external environmental magnetic fields. The sensor head and Dewar (not shown) are preferably fixed in space, the Dewar more preferably resting on the ground within the Faraday cage, and in communication with the SQUID electronics (not shown), which are preferably located outside the Faraday cage 10. The patient support device 14 includes a seat portion 16 and a back portion 18. The patient support device 14 is rotatable 22 at least a full 360°, with the back portion 18 being reclinable 24, preferably from a vertical position to a position about 45° from vertical. The patient support device is also controlled horizontally 26 and vertically 28 in order to maintain the sensor head in contact with the head of the test subject 50 as the angle of inclination of the patient support device back 18 is changed or the patient support device is rotated. The patient support device 14 also includes a head stabilizer 30 to maintain the head in a predetermined fixed position with respect to the patient support device back. The head stabilizer 30 preferably contacts the cheeks of the test subject 50 to immobilize the cheek bones, thereby immobilizing the head.

The vertical, horizontal, rotational, and recline adjustments to the support device may be manual or automated. In some embodiments, the SQUID electronics includes a monitor and a computer with software for operation of the SQUID sensors and control of the position of the patient support device 14. In other embodiments, the vertical, horizontal, rotational, and recline adjustments are done manually, and a sensor is used to determine the location of the test subject's head surface with respect to the SQUID sensor.

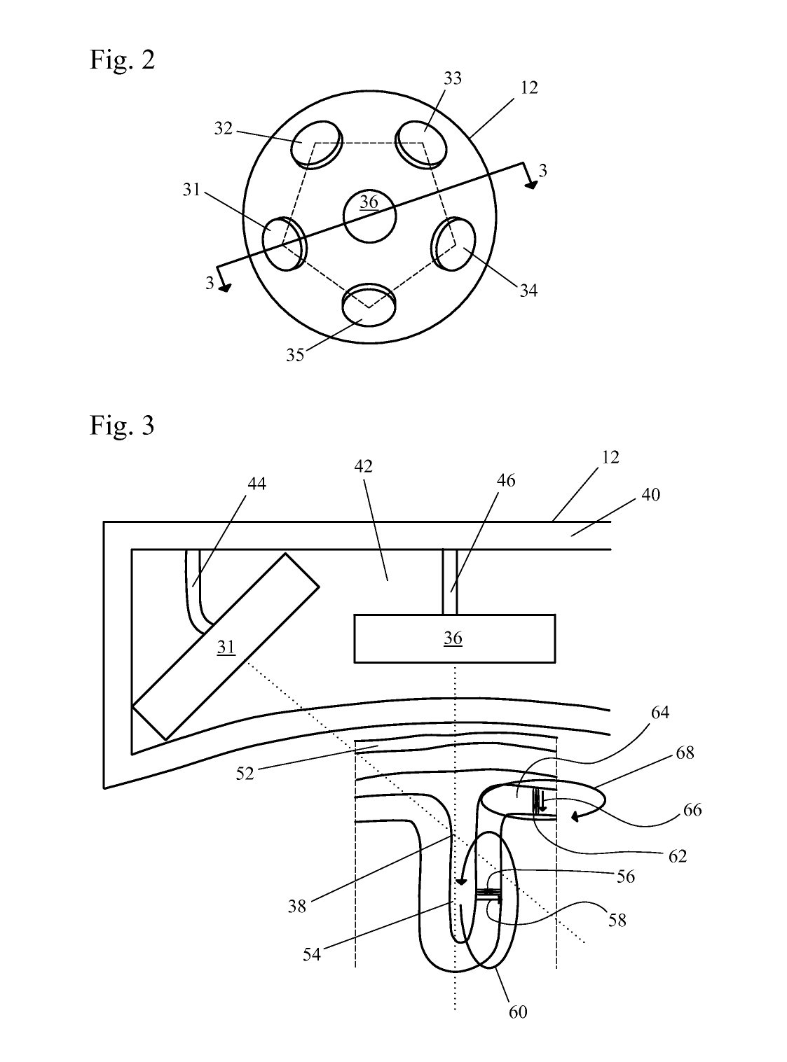

FIG. 2 shows a top view of the SQUID sensor head 12 with five SQUID sensors 31, 32, 33, 34, 35 in an array around a sixth central SQUID sensor 36. The central SQUID sensor 36 is flat with the five surrounding SQUID sensors 31, 32, 33, 34, 35 oriented at a fixed angle toward the central SQUID sensor 36. The fixed angle in FIG. 2 is about 45°.

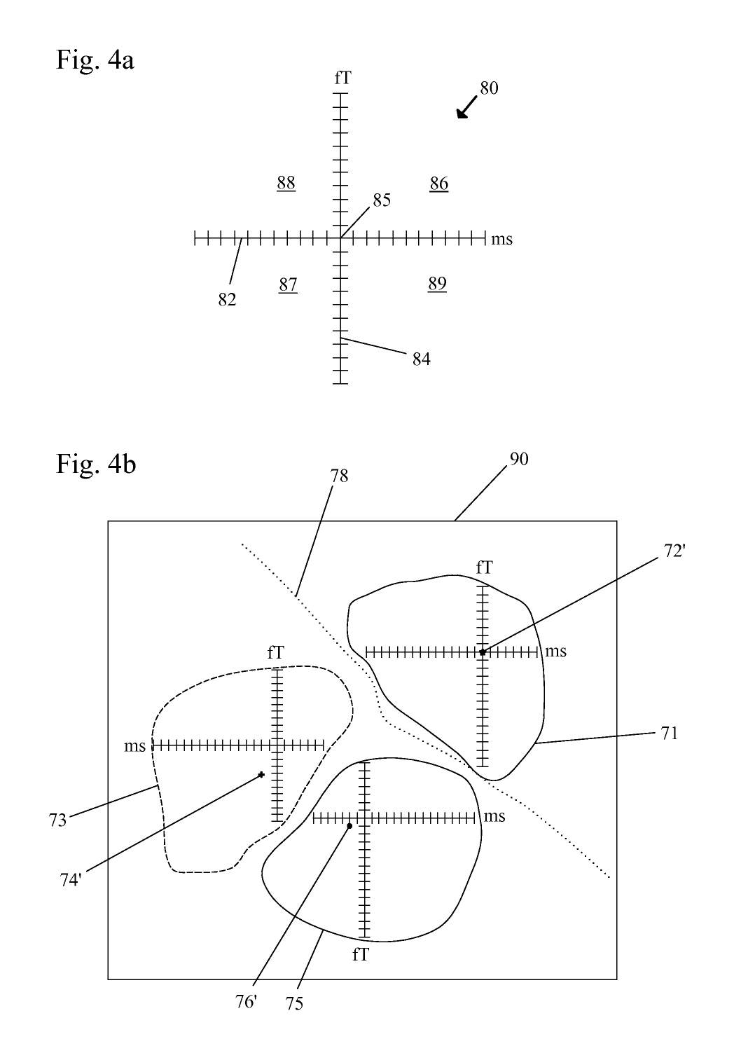

FIG. 3 shows the SQUID sensor head 12 placed against the scalp 52 of the test subject 50 above a sulcus 54 of interest. The peripheral sensors (only one 31 shown in FIG. 3) and the central sensor 36 all converge on a focal point 38 about two to four centimeters below the central sensor 36. The sensor head 12 includes a Dewar housing 40 for the sensors. The Dewar housing 40 holds the liquid helium in the enclosed portion 42 of the sensor head 12 to maintain the SQUID sensors 31, 32, 33, 34, 35, 36 at superconducting temperatures and insulates the sensors and liquid helium from the environment and the head of the test subject. Electrical wiring 44, 46 powers each of the SQUID sensors. The neuronal structures 56, and hence the electrical impulses, in the sulcal wall are oriented substantially parallel 58 to the scalp 52, thereby generating a magnetic field 60 in a plane substantially perpendicular to the scalp 52. In contrast, the neuronal structures 62, and hence the electrical impulses, of the gyms 64 are oriented substantially perpendicular 66 to the scalp 52, thereby generating a magnetic field 68 in a plane substantially parallel to the scalp 52. The magnetic field 60 generated from electrical activity in the sulcus 54 therefore is much more easily detected than the magnetic field 68 generated from electrical activity in the gyms 64 with the sensor head 12 located as shown in FIG. 3.

FIG. 4b through FIG. 4d show idealized sample test results from a SQUID system. After determining the relative maximum field strength and latency and comparing the generated magnetic fields for the specific test subject to average generated magnetic fields for the “normal” population or generated magnetic fields from the specific test subject from the same test performed at an earlier time, the peaks may be plotted on a graph 80 such as the one shown in FIG. 4a for a single magnetic field. The x-axis 82 represents the latency in milliseconds (ms), and the y-axis 84 represents the absolute value of the field strength in femtotesla (fT). A magnetic field with a peak having a field strength equal to the field strength of a normal test subject and a latency corresponding to a normal latency is plotted as a point at the origin 85. An abnormally rapid and strong field transmission is plotted in the upper right quadrant 86, whereas a delayed transmission time with a weak field transmission is plotted in the lower left quadrant 87. A delayed transmission time with a strong field transmission is plotted in the upper left quadrant 88. An abnormally rapid and weak field transmission is plotted in the lower right quadrant 89.

FIG. 4b shows a display 90 representing the field strength and latency of the maximum peaks as points 72′, 74′, 76′ of the three fields of FIG. 4d. In the display 90 of FIG. 4b, the spatial relationship of the three fields has been preserved, and one of the isofields 71, 73, 75 is shown for each point 72′, 74′, 76′, respectively. The grid for each field is overlaid and positioned based on the calculated relative maximum field strength and latency. For FIG. 4b, the points 72′, 74′, 76′ are determined based on the field strength and latency from collected data across all monitored frequencies. FIG. 4b shows that the maximum negative polarity of the left field occurred with a maximum field strength that was 4 fT below “normal” as indicated by point 74′ and the maximum positive polarity of the bottom field occurred with a maximum field strength that was 1 fT below “normal” as indicated by point 76′. Both maximum peaks occurred at a time 2 ms slower than “normal”. The maximum positive polarity of the upper right field occurred with a “normal” maximum field strength at a “normal” time as indicated by point 72′. This indicates normal activity in the brain where the first peak 72′ occurs but some type of abnormality in the brain where the last two peaks 74′, 76′ occur. The line 78 between the normal and abnormal results represents a potential limit of the abnormality. Hence, the graph provides an easily-understood summary of the results of a stimulus test, including the relative strengths and latencies of multiple magnetic fields generated at different locations and different times after the stimulus event, on a single display. Alternatively, the display may show only one single formatting grid at a time.

To produce the display shown in FIG. 4c, similar comparisons are made as in the comparisons made to produce the data of FIG. 4b, except that the test data has been separated by frequencies among frequency ranges as opposed to including the entire frequency range of collected data. The frequency ranges are preferably the previously-described δ, θ, α, β, γ, and Hγ frequency ranges, but alternative frequency ranges may be used within the spirit of the present invention. Depending on the type of stimulus and the portion of the brain being monitored, data in only one or several of the frequency ranges may be relevant. In some embodiments, only the frequency ranges with data that deviates from the norm or from a previous test result are plotted as points on the display. FIG. 4c shows a display 100 representing the field strength and latency of the maximum individual frequency range peaks of the three fields of FIG. 4d.

In the display 100 of FIG. 4c, the spatial relationship of the three fields has been preserved, and one of the isofields 71, 73, 75 is shown for each field. In the upper right field, the field strength and latency is normal for all of the frequency ranges, as indicated by the point 101 at the origin. In the left field, points for α 102, β 103, and γ 104 frequency ranges are shown. In the bottom field, points for α 106, β 107, and γ 108 frequency ranges are shown. In both the left field and the bottom field, the different frequency ranges have different maximum field strength and latency deviations. The points for the different frequency ranges are preferably consistently identified in a predetermined manner such that a user can visually identify what frequency range is being represented by a given point. The points preferably have different shapes or colors to identify to which frequency range they belong. The points more preferably are in the form of the Greek letter of the frequency range represented, as shown in FIG. 4c.

In FIG. 4d, a two-dimensional image shows magnetic isofield lines 71, 73, 75 at a certain time point after a predetermined stimulus event. This is a traditional snapshot representation of raw data at one time point before any kind of comparison with previous test results or normal results. The solid lines 71, 75 represent positive polarity field strengths and the dashed lines 73 represent negative polarity field strengths. The images shows three peaks 72, 74′, 76′, two with a positive polarity 72, 76′ and one with a negative polarity 74′. At the time of the sample data shown in FIG. 4d, the left field and the bottom field are at their maximum strength, but the upper right field is not.

In some embodiments, the formatting grid shows results for a single generated magnetic field from repetition of the same type of stimulus test on the same test subject at different times. Arrows may be used to indicate that the test subject's results are trending toward normal over time between the tests, indicating that the condition causing the abnormality in the test subject is improving or the treatment regimen is working, or away from normal, indicating that the condition causing the abnormality is worsening. The improvements may, for example, be the result of a cumulative effect of treatment of the test subject with one or more pharmaceuticals over time or may be the result of increasing the dose taken by the test subject of one or more pharmaceuticals between tests.

The sulcus location may be localized directly from the SQUID signals. For example, when the right index finger is stimulated, the SQUID signal maximum is over the left sensory cortex, where sensory input from the finger is registered (see, for example, Hamälainen et al., “Magnetoencephalography—theory, instrumentation, and applications to noninvasive studies of the working human brain”, Rev. Mod. Phys., Vol. 65, pp. 413-497, 1993).

More generally, the sulcus represents an absolute limit to current transmission and thus to magnetic field. That is, a sensor placed contralateral to a sulcus-generated signal detects signals from, effectively, a point source and the signal strength decreases as the inverse cube of the distance from the source. A sensor placed ipsilateral to a sulcus-generated signal has characteristics of a dipole such that the signal strength decreases as the inverse square of the distance from the source. The detectors contralateral to the gyms of interest should demonstrate a decay in intensity as the cube function of distance. The output should be markedly simplified for interpretation but not degraded.

Using software that requires a signal to show predicted attenuation by all of the sensors, it is estimated that>>99% of the signals will be rejected. For a signal to be scored, the signal must demonstrate a strength decay for all detectors as a function of distance from a local source, i.e., beneath the brain area covered by the detector and below the detector to a distance of about 3 cm. Any signal of common intensity detected by the array is rejected. Thus, many signals of brain origin distal to the subjacent cortical region of interest are also rejected. With current full brain MEG imaging, this strategy is not done and may not be possible. Recent efforts, because of the enormous signal density of complete brain imaging, have developed only a method to distinguish signals originating within the brain from signals originating outside the brain (see, for example, Taulu et al., “Removal of Magnetoencephalographic Artifacts With Temporal Signal-Space Separation: Demonstration With Single-Trial Auditory-Evoked Responses”, Human Brain Mapping, Vol. 30, pp. 1524-1534, 2009).

A functional aspect of the brain organization adds to the utility of the present systems and methods. The brain function is often delimited by the sulcus. That is to say, across the major sulcus from the sensory region corresponding to the finger lies the motor cortex with totally different function. On reflection, this feature may exist to prevent undesirable propagation of signals. For example, if the finger sensory region stimulated not just the sensory cortex but also the motor cortex, the finger would be directed to contract and the ascending sensory pathways would return the signal to the sensory cortex. A non-terminating circuit would be completed.

The systems and methods, by imaging the sulcus, provide functional information from two brain regions on either side of the sulcus with very different functions. This information enriches the output values. If there is a signal change from normal baseline, either it is a feature of regional change affecting function on both sides of the sulcus or the change is restricted to a certain neural pathway.

The systems and methods are used to create a regional magnetic cortical surface map to inventory the function of hearing, sight, touch, movement, and cognition of a normal healthy brain. This information would allow the analysis of individuals in disease states or other conditions of interest.

In other embodiments, an array of three to nine or possibly more SQUID sensors about one centimeter in size with a fixed radial geometry images the surface of the brain via a computer-directed movable C-arm. C-arms are currently used commonly in x-ray machines with one end of the C-shaped structure generating the x-ray radiation and the other end holding the x-ray detector. In the present system, each sensor in the array can function as an axial gradiometer to attenuate the environmental magnetic noise. In one embodiment, the position of the array is correlated by ultrasound imaging of the head to give a precise array location relative to the brain structures and thus generates a functional map of the sulcal cortical surface. Only signals that demonstrate the expected strength decay laterally between sensors that is consistent with a superficial signal origin are scored. Software directs the movable sensor array to refine the image in order to provide a robust surface map of the surface sulcal activity, thereby specifically creating a map of basal neural activity or “noise”.

In other embodiments, the systems and methods are used to detect abnormalities in a subject human brain by comparing the magnetic cortical surface map of the subject human to the magnetic cortical surface map of a normal healthy brain.

In yet other embodiments, the systems and methods are used to detect temporal changes in a magnetic cortical surface map as a result of application of one or more controlled stimuli to a human subject as described above. In some of these embodiments, the results are used to give a better understanding of the correlation between stimuli and human brain activity. As described, recent investigations have used EEG to distinguish between normal individuals and those in a chronic vegetative state. In response to a sound stimulus, individuals in a vegetative state had a signal in the superior temporal gyrus cortex but not a subsequent signal in the inferior frontal gyrus cortex. In normal individuals the “top down” signal originating in the inferior frontal gyms is preserved. The EEG system was able to distinguish between the normal and minimally alert individuals (see Boly et al.). As the accompanying review noted, improved methods are “sorely needed” (Miller, “Feedback From Frontal Cortex May Be a Signature of Consciousness”, Science, Vol. 332, p. 779, 2011).

There is evidence already for individuals with autism evaluated with MEG imaging that there is delay in latency but no change in field strength (see Roberts et al., “MEG Detection of Delayed Auditory Evoked Responses in Autism Spectrum Disorders: Towards an Imaging Biomarker for Autism”, Autism Research, Vol. 3, pp. 8-18, 2010).

All above-mentioned references are hereby incorporated by reference herein.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of evaluating electrical activity in at least a portion of a brain comprising a cerebral cortex having a surface with a plurality gyri separated by sulci, the method comprising the steps of:
a) a computer imaging the brain to determine a geometry of the surface of the cerebral cortex;
b) the computer measuring a plurality of magnetic field strengths around the brain in a plurality of locations using at least one array of a plurality of sensors of at least one superconducting quantum interference device;
c) the computer evaluating the magnetic field strengths measured by the sensors to quantify at least one magnetic field generated by electrical activity in the brain; and
d) the computer localizing the magnetic field generated by electrical activity in the brain to at least one location on the surface of the cerebral cortex to generate a magnetic topological cortical surface map.

2. The method of claim 1, wherein step b) is performed for a single time point.

3. The method of claim 1, wherein step b) is performed for a plurality of time points.

4. The method of claim 3 further comprising the step of the computer stimulating the brain with at least one stimulus.

5. The method of claim 4, wherein the at least one stimulus is selected from the group consisting of at least one visual stimulus, at least one auditory stimulus, at least one tactile stimulus, at least one cognition stimulus, and any combination of these.

6. The method of claim 4 further comprising the step of the computer comparing a response by the brain to a known response by a normal brain.

7. The method of claim 4 further comprising the step of the computer comparing a response by the brain to a known response by an abnormal brain with a known abnormal condition.

8. The method of claim 1, wherein the computer localizes the magnetic field only to one or more of the sulcal-defined gyri of the surface of the cerebral cortex.

9. The method of claim 1 further comprising the step of comparing the magnetic cortical surface map to a reference magnetic cortical surface map.

10. A superconducting quantum interference device comprising:
an array of superconducting quantum interference device detectors, wherein the detectors in the array are at a fixed angle with respect to an axis perpendicular to a plane of the array; and
a processor receiving data from the detectors and determining magnetic fields generated within about five centimeters of the detectors based on magnetic field decay based on inverse square decay with distance ipsilateral to a sulcus and inverse cube decay with distance contralateral to the sulcus.

11. A non-paramagnetic support device for a test subject comprising:
a seat portion having a substantially horizontal seat surface supporting the weight of the test subject;
a back portion extending upward from the seat portion, the back portion having a back surface supporting a back of the test subject, a back angle between the back surface and the substantially horizontal seat surface being adjustable by an angular adjustment mechanism;
a rotation adjustment mechanism rotating the seat portion around a vertical axis;
a horizontal adjustment mechanism translating the seat portion in a horizontal plane;
a vertical adjustment mechanism elevating the seat portion in a vertical plane; and
a processor evaluating a position of the back portion based on positions of the angular adjustment mechanism, the rotational adjustment mechanism, the horizontal adjustment mechanism, and the vertical adjustment mechanism and directing the angular adjustment mechanism, the rotational adjustment mechanism, the horizontal adjustment mechanism, and the vertical adjustment mechanism to change the position of the support device.

12. The non-paramagnetic support device of claim 11 further comprising a head stabilizer extending from the back portion, the head stabilizer immobilizing a head of the test subject to a predetermined position.

13. The non-paramagnetic support device of claim 11, wherein the back angle is adjustable within a range of a vertical position and a fixed angle with respect to vertical.

14. The non-paramagnetic support device of claim 11, wherein the rotation mechanism permits at least 360° of rotation of the seat portion.

15. A non-paramagnetic support device for a test subject comprising:
a seat portion having a substantially horizontal seat surface supporting the weight of the test subject;
a back portion extending upward from the seat portion, the back portion having a back surface supporting a back of the test subject, a back angle between the back surface and the substantially horizontal seat surface being manually adjustable;
a head stabilizer extending from the back portion, the head stabilizer immobilizing a head of the test subject to a predetermined position with respect to the back portion;
a sensor determining a position of the head of the test subject;
wherein the seat portion, the back portion, and the head stabilizer are horizontally, vertically, and rotationally manually adjustable.

16. The non-paramagnetic support device of claim 15, wherein the back angle is adjustable within a range of a vertical position and a fixed angle with respect to vertical.

17. The non-paramagnetic support device of claim 15, wherein the seat portion, the back portion, and the head stabilizer are rotationally adjustable by at least 360°.

18. A method of displaying electrical activity in at least a portion of a brain based on at least one magnetic field generated by the electrical activity, the method comprising the steps of:
a computer detecting the magnetic field with an array of SQUID sensors centered above a sulcus of the brain; and
the computer graphically displaying the electrical activity based on the magnetic field as a contour map;
wherein closed curves on the contour map represent points of equal potential.

19. The method of claim 18 further comprising displaying a change in electrical activity with respect to time in slow motion with respect to the timing of the electrical activity.

20. The method of claim 18, wherein a height of a stack of the closed curves represents an intensity of the measured field.

21. A method of displaying electrical activity in at least a portion of a brain based on at least one magnetic field generated by the electrical activity, the method comprising the steps of:
a computer detecting the magnetic field with an array of SQUID sensors centered above a sulcus of the brain;
the computer determining a test maximum field strength at a test latency after a stimulus event for the magnetic field;
the computer comparing the test maximum field strength and the test latency to a normal maximum field strength and a normal latency for a normal subject for the stimulus event; and
the computer displaying on a screen a data point representing the test maximum field strength and the test latency on a two-dimensional formatting grid having maximum field strength on a first axis and time on a second axis and an origin representing the normal maximum field strength and the normal time such that the data point not being located at the origin indicates a deviation in brain activity data of the test subject relative to a normal subject.

22. A method of detecting electrical activity in at least a portion of a brain based on a magnetic field generated by the electrical activity, the method comprising the steps of:
a computer detecting the magnetic field with an array of SQUID sensors centered above a sulcus of the brain based on detection of frequencies within a range of about 0 Hertz to about 100 Hertz.

23. The method of claim 22 further comprising the steps of:
the computer scoring a plurality of signals received by the SQUID sensors based on calculated decay characteristics for all the SQUID sensors as a function of distance from a local source in the brain; and
the computer filtering out signals determined to be originating outside the local source of the brain comprising the computer rejecting signals showing a common intensity for all of the SQUID sensors.

24. A method of displaying comparative electrical activity in at least a portion of a brain of a test subject based on at least one magnetic field generated by the electrical activity, the method comprising the steps of:
a computer detecting the magnetic field as brain activity data with an array of SQUID sensors centered above a sulcus of the brain after a stimulus event; and
the computer determining a test maximum field strength at a test latency after the stimulus event for the brain activity data for at least a current portion of the brain activity data;
the computer comparing the test maximum field strength and the test latency of the current portion of the brain activity data to a previous maximum field strength and a previous latency of an analogous portion of brain activity data of the test subject for the stimulus event acquired at an earlier time; and
the computer displaying on a screen a data point representing the test maximum field strength and the test latency on a two-dimensional formatting grid having comparative maximum field strength on a first axis and comparative latency on a second axis and an origin representing the previous maximum field strength and the previous latency such that the data point not being located at the origin indicates a change in brain activity data of the test subject since the earlier time.
